SEQUENCE LISTING (3 sequences)

SEQ ID NO: 1 - DNA, 2226 bp, synthetic construct
  source 1..2226: mol_type = other DNA
atggctctcttcactccgtggaagttgtcctctcagaagctgggctttttcctggtgacttttggcttcatttggggtatgatgcttctgcactttaccatccagcagcgaactcagcctgaaagcagctccatgctgcgcgagcagatcctggacctcagcaaaaggtacatcaaggcactggcagaagaaaacaggaatgtggtggatgggccatacgctggagtcatgacagcttatgatctgaagaaaacccttgctgtgttattagataacattttgcagcgcattggcaagttggagtcgaaggtggacaatcttgttgtcaatggcaccggaacaaactcaaccaactccactacagctgttcccagcttggttgcacttgagaaaattaatgtggcagatatcattaacggagctcaagaaaaatgtgtattgcctcctatggacggctaccctcactgtgagggaaagatcaagtggatgaaagacatgtggcgttcagatccctgctacgcagactatggagtggatggatccacctgctctttttttatttacctcagtgaggttgaaaattggtgtcctcatttaccttggagagcaaaaaatccctacgaagaagctgatcataattcattggcggaaattcgtacagattttaatattctctacagtatgatgaaaaagcatgaagaattccggtggatgagactacggatccggcgaatggctgacgcatggatccaagcaatcaagtccctggcagaaaagcagaaccttgaaaagagaaagcggaagaaagtcctcgttcacctgggactcctgaccaaggaatctggatttaagattgcagagacagctttcagtggtggccctcttggtgaattagttcaatggagtgatttaattacatctctgtacttactgggccatgacattaggatttcagcttcactggctgagctcaaggaaatcatgaagaaggttgtaggaaaccgatctggctgcccaactgtaggagacagaattgttgagctcatttacattgatattgtaggacttgctcaattcaagaaaactcttggaccatcctgggttcattaccagtgcatgctccgagtccttgattcatttggtactgaacccgaatttaatcatgcaaattatgcccaatcgaaaggccacaagaccccttggggaaaatggaatctgaaccctcagcagttttataccatgttccctcataccccagacaacagctttctggggtttgtggttgagcagcacctgaactccagtgatatccaccacattaatgaaatcaaaaggcagaaccagtcccttgtgtatggcaaagtggatagcttctggaagaataagaagatctacttggacattattcacacatacatggaagtgcatgcaactgtttatggctccagcacaaagaatattcccagttacgtgaaaaaccatggtatcctcagtggacgggacctgcagttccttcttcgagaaaccaagttgtttgttggacttgggttcccttacgagggcccagctcccctggaagctatcgcaaatggatgtgcttttctgaatcccaagttcaacccacccaaaagcagcaaaaacacagactttttcattggcaagccaactctgagagagctgacatcccagcatccttacgctgaagttttcatcgggcggccacatgtgtggactgttgacctcaacaatcaggaggaagtagaggatgcagtgaaagcaattttaaatcagaagattgagccatacatgccatatgaatttacgtgcgaggggatgctacagagaatcaatgctttcattgaaaaacaggacttctgccatgggcaagtgatgtggccacccctcagcgccctacaggtcaagcttgctgagcccgggcagtcctgcaagcaggtgtgccaggagagccagctcatctgcgagccttctttcttccagcacctcaacaaggacaaggacatgctgaagtacaaggtgacctgccaaagctcagagctggccaaggacatcctggtgccctcctttgaccctaagaataagcactgtgtgtttcaaggtgacctcctgctcttcagctgtgcaggcgcccaccccaggcaccagagggtctgcccctgccgggacttcatcaagggccaggtggctctctgcaaagactgcctatag

SEQ ID NO: 2 - DNA, 34 bp, synthetic construct
  source 1..34: mol_type = other DNA
acggtaccaccatggctctcttcactccgtggaa

SEQ ID NO: 3 - DNA, 33 bp, synthetic construct
  source 1..33: mol_type = other DNA
acctcgagctataggcagtctttgcagagagcc

(19) United States
(12) Patent Application Publication
(10) Pub. No.: US 2024/0166711 A1
(43) Pub. Date: May 23, 2024

(54) Method for promoting the stemness and/or transdifferentiation of acinar cells

(71) Applicant: National Taiwan University, Taipei City (TW)

(72) Inventors: Pei-Jen Lou, Taipei City (TW); Tai-Horng Young, Taipei City (TW); Ching-Chia Cheng, Taipei City (TW); Mei-Chun Lin, Taipei City (TW); Hisn-Lin Chen, New Taipei City (TW)

(21) Appl. No.: 18/057,051

(22) Filed: Nov. 18, 2022

(51) Int. Cl. C07K 14/625; C12N 5/071; C12N 9/10; C12N 15/86

(52) U.S. Cl. CPC C07K 14/625; C12N 5/0633; C12N 9/1051; C12N 15/86; C12Y 204/01155; C12N 2510/02; C12N 2740/15043; C12N 2740/15052

(57) ABSTRACT

The present application provides a method for promoting the sternness and/or transdifferentiation of acinar cells, comprising the following steps: providing an acinar cell, transfecting a plasmid into the acinar cell, and culturing the transfected acinar cell, wherein the plasmid contains a genetic material for overexpression of N-acetylglucosaminyltransferase V (GnT-V).

### INCORPORATION BY REFERENCE OF SEQUENCE LISTING PROVIDED AS AN XML FILE

A Sequence Listing is provided herewith as a Sequence Listing XML, “20221125-US20221012-Sequence listing” created on Nov. 24, 2022 and having a size of 6 KB. The contents of the Sequence Listing XML are incorporated by reference herein in their entirety.

### BACKGROUND OF THE INVENTION

### Field of the Invention

The present invention relates to a method for promoting the sternness and/or transdifferentiation of acinar cells, particularly a method for transdifferentiating the acinar cells into insulin-secreting cells.

### Descriptions of the Related Art

Saliva is mainly secreted by parotid gland acinar cells (parotid gland acinar cells, ACs) to keep the health of the oral cavity. However, irreversible damage to salivary glands is a side effect of radiation treatment for head and neck cancer. Up to now, there is no effective therapy for persistent salivary hypofunction (V. W. C. Wu & Leung, Frontiers in Oncology, 9, 1090, 2019). Approximately 90% of the glandular volume is constituted by acinar cells (ACs), which are mainly responsible for the synthesis and secretion of salivary components (Ambudkar, Cell Calcium, 55, 297-305, 2014; Pedersen et al., Journal of Oral Rehabilitation, 45, 730-746, 2018; Williams & Cope, Journal of Anatomy, 116, 431-444, 1973). Thus, the demand for ACs is gradually increasing for biomedical research to treat or replace destroyed glands. Freshly isolated human parotid gland ACs are the best choice for such applications. Previously, we developed a stable protocol to harvest ACs with high purity from human parotid gland tissues (Chan et al., Head and Neck, 33, 407-414, 2011). However, it is difficult to enrich the amount of ACs for further applications since the lack of a regular supply of salivary gland specimens and the loss of the replicative ability of ACs in vitro.

Besides, it has been reported that pancreatic ACs can transdifferentiate into insulin-secreting cells with secretory properties (Minami et al., Proceedings of the National Academy of Sciences of the United States of America, 102, 15116-15121, 2005; Song et al., Biochemical and Biophysical Research Communications, 316, 1094-1100, 2004), but whether salivary acinar cells can be transdifferentiated to produce insulin remains unclear. Patients with diabetes receive insulin orally or parenterally at a fixed time in current treatment. Hence, there is still lack of an insulin-producing system to reduce the frequency for oral or parenteral administration of insulin.

### SUMMARY OF THE INVENTION

In view of the weaknesses of the prior art, the present invention provides a method for promoting the stemness and/or transdifferentiation of acinar cells, comprising the following steps:
providing an acinar cell,
transfecting a plasmid into the acinar cell, and
culturing the transfected acinar cell,
wherein the plasmid contains a genetic material for overexpression of N-acetylglucosaminyltransferase V (GnT-V).

In an embodiment, the plasmid is transfected into the acinar cell by calcium phosphate, liposome, viral vector or electroporation. In particular, the viral vector is lentiviral vector. In an embodiment, the genetic material contains MGAT5 cDNA, the sequence set forth as SEQ ID NO: 1. In particular, the MGAT5 cDNA is derived by amplifying with primers of SEQ ID NO: 2 and SEQ ID NO: 3. In an embodiment, the transfected acinar cell can transdifferentiate into an insulin-secreting cell. In an embodiment, the acinar cell is a salivary gland acinar cell. In particular, the salivary gland acinar cell is a parotid gland acinar cell. In addition, the present invention further provides an insulin-secreting cell obtained by using the abovementioned method, and a method for producing insulin by using the above method, which further comprises a step of harvesting insulin from the cell culture medium. The sternness of acinar cells can be promoted through the method disclosed in the present invention. It can be applied to tissue engineering of the human salivary gland to treat patients with head and neck cancer after radiotherapy. Also, it can be used as a novel insulin production system to enable the parotid gland tissue of diabetic patients to synthesize insulin by itself and regulate blood sugar concentration.

### BRIEF DESCRIPTION OF DRAWINGS

FIG. 1 is the mRNA expression of sternness-related genes of the ACs/GnT-V.

FIG. 2 is the expressions of Sox2, Oct3/4, and Nanog on the cells surface of the ACs/GnT-V.

FIG. 3 is the expressions of CD90, and CD49f on the cells surface of the ACs/GnT-V.

FIG. 4 is the expressions of α-amylase, AQP3, and AQP5 in cellular of the ACs/GnT-V.

FIG. 5A is the cell proliferation of passage 1 of the ACs/GnT-V.

FIG. 5B is the cell proliferation of passage 2 of the ACs/GnT-V.

FIG. 5C is the cell proliferation of passage 3 of the ACs/GnT-V.

FIG. 6 is the population doublings of the ACs/GnT-V.

FIG. 7 is the motility and invasiveness of the ACs/GnT-V.

FIG. 8 is the expressions of insulin in cellular of the ACs/GnT-V.

FIG. 9 is the expressions of insulin secreted by the ACs/GnT-V.

FIG. 10 is the result of the insulin receptor activated by the secreted insulin of the ACs/GnT-V.

FIG. 11 is the result of the glucose uptake increased by the secreted insulin of the ACs/GnT-V.

### DETAILED DESCRIPTION OF THE INVENTION

Reference will be made in detail description to the exemplary embodiments and drawings for being more readily understood to the advantages and features of the present invention, as well as the methods of attaining them. However, the present invention may be carried out in many different forms and should not be construed as limited to the embodiments set forth herein. Conversely, these embodiments are provided to render the present disclosure to be conveyed the scope of the present invention more thoroughly, completely, and fully to one having ordinary skill in the art of the present invention. Moreover, the present invention would be defined only by the appended claims. As used herein, the term “and/or” includes any and all combinations of one or more of the associated listed components.

Unless otherwise defined, all terms (including technical and scientific terms) used herein have the same meaning as generally understood by one having ordinary skill in the art of the present invention. It will be more understandable that, for example, the terms defined in commonly used dictionaries should be understood to have meanings consistent with the contents of the relevant fields, and would not be interpreted overly idealized or overly formal unless clearly defined herein. As described in the present specification, a range of values is used as a shorthand to describe each and every numerical value in the range, and any number within that range may be chosen as the end-value of that range.

Several examples are listed below as exemplifications to illustrate the implementation of the present application. Those skilled in the art can easily understand the advantages and effects that this invention can achieve through the content of this specification, and carry out without departing from the spirit of this invention. Various modifications and changes to implement or apply the content of this invention, other similar or equivalent methods and materials described in this article to implement or test this application, should be considered as covered by this application.

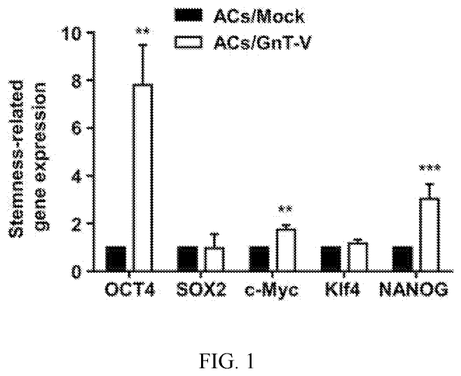

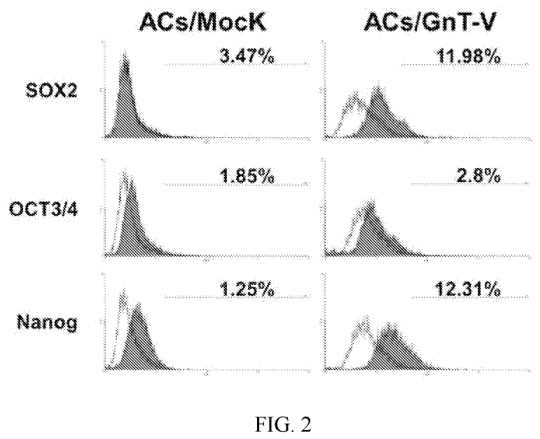

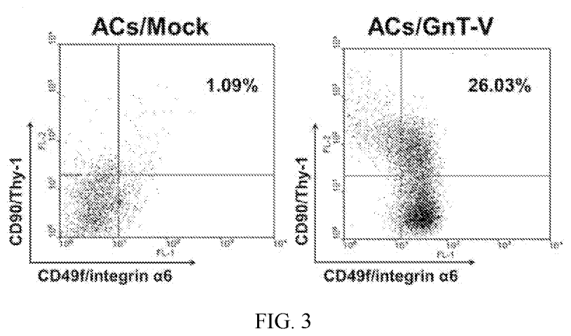

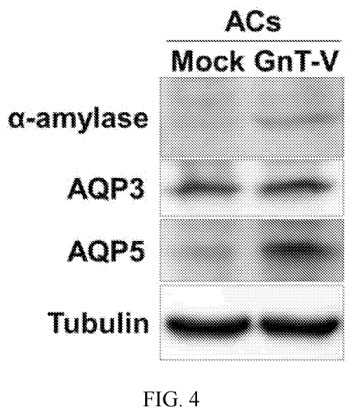

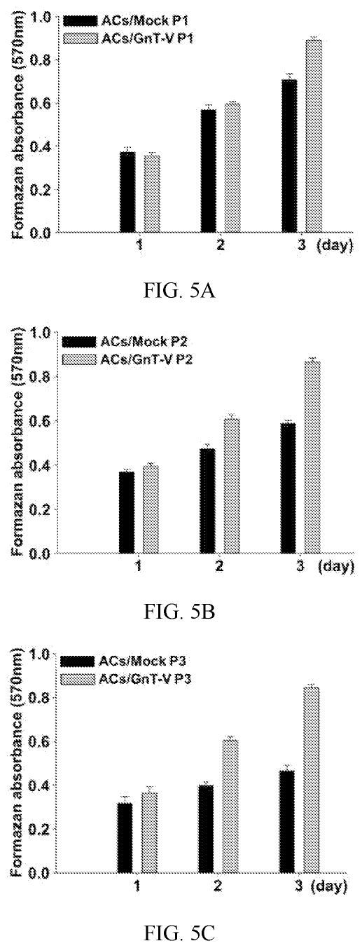

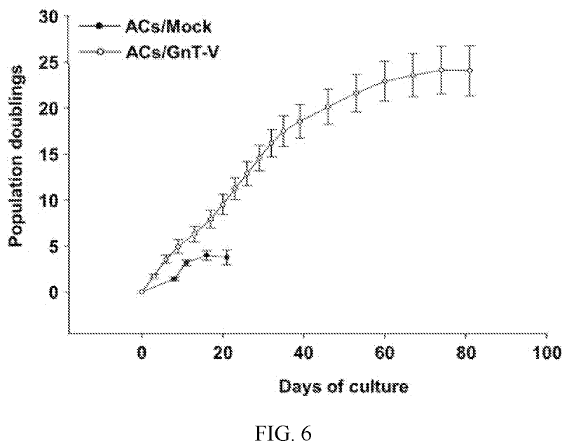

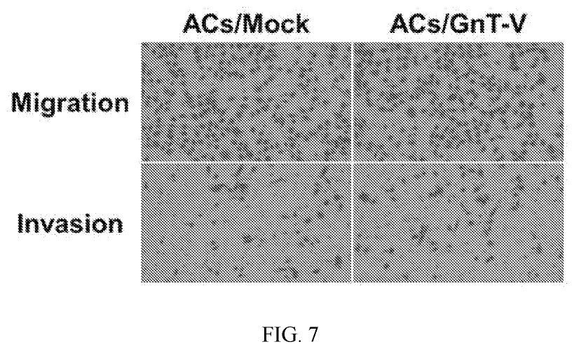

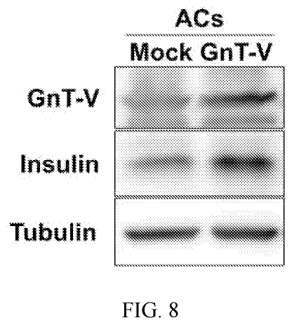

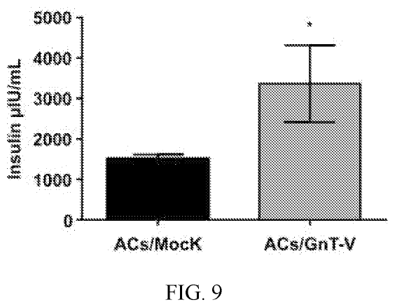

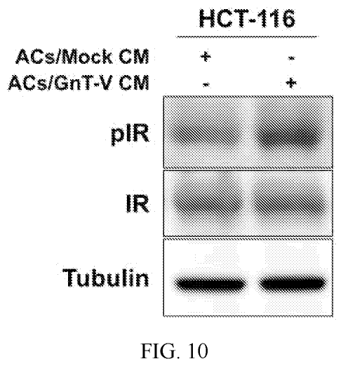

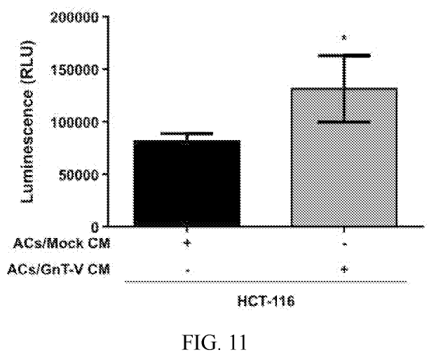

As referred to herein, “acinar cells” can be isolated from the pancreas and salivary glands. Salivary gland acinar cells are selected to use in the following examples, particularly the parotid gland acinar cells in salivary gland. The study procedures were approved by the Institutional Review Board of National Taiwan University Hospital. 250 donors' parotid samples were recruited from 2019 to 2021, and the parotid gland tissues were obtained from patients during surgery. The human specimens were stored in cold phosphate-buffered saline (PBS) and washed twice with ice-cold PBS to remove blood or other undesirable substances. The isolated shallow-yellow acinar tissues from parotid gland tissue were treated with collagenase B (Roche Diagnostics GmbH) and DNase I (Roche) at 37° C. for 120 min. The digested tissue solution was filtered by a 100 μm cell strainer and centrifuged at 1200 rpm for 5 min Finally, the supernatant was discarded, the ACs were cultured in keratinocyte serum-free medium (Invitrogen) containing 50 mg/ml bovine pituitary extract and 5 ng/ml human recombinant epidermal growth factor and incubated at 37° C. in a humidified 5% CO₂ atmosphere. The detailed steps are described in the paper “Salivary gland acinar cells spontaneously form three-dimensional structures and change the protein expression patterns” by Chan et al. in the Journal of Cellular Physiology Human (226, 3076-3085) in 2011. The contents of the paper are incorporated by reference in their entireties herein. Example 1—Preparation of the Plasmid The linear DNA fragment encoding GnT-V was amplified from a previously cloned MGAT5 complementary DNA (SEQ ID NO: 1) with the sense primer of SEQ ID NO: 2 (5′-ACGGTACCACCATGGCTCTCTTCACTCC GTGGAA-3′) and the anti-sense primer of SEQ ID NO: 3 (5′-ACCTCGAGCTATAGGCAGTCTTTGCAGAGAGCC-3′), in which the KpnI and XhoI sites were introduced. The sense primer included an additional Kozak consensus sequence with an ATG initiation codon for proper initiation of translation. The PCR was carried out with 2.5 U of Pfu DNA polymerase in a final volume of 50 μl using the following conditions: 94° C. for 5 min, 30 cycles (94° C. for 45 s, 65° C. for 30 s, and 72° C. for 2 min) and a final extension of 72° C. for 5 min. The PCR product was purified by agarose gel extraction and digested by KpnI and XhoI, and then cloned into pENTR™3C dual selection vector to yield pENTR3C/GnT-V plasmid. The plasmid can be introduced into cells by conventional transfection protocol, e.g., transfected into the acinar cells by calcium phosphate, liposome, viral vector, or electroporation. The lentiviral vector is used as an example below. The pENTR3C/GnT-V plasmid is recombined into pLenti6/V5-DEST to obtain pLenti6/V5-DEST/GnT-V plasmid. Example 2—Preparation of the lentiviral vectorpLenti6/V5-DEST/GnT-V was co-transfected with the envelope plasmid (pMD2.G) and the packaging plasmid (pCMVdeltaR8.91) into HEK293T cells using Lipofectamine 3000 (Invitrogen, Waltham, Massachusetts, United States) according to the manufacturer's instructions. The viruses were harvested from the culture medium on Day 2 after transfection and filtered with a filter of 0.22 μm or 0.45 μm. The obtained lentivirus can be used to infect acinar cells by conventional protocol to transfect the plasmid that can overexpress GnT-V into acinar cells. Example 3—sternness determinations of GnT-V-overexpressing acinar cells For determining the sternness properties of the transfected acinar cells (ACs/GnT-V), the mRNA expression of OCT4, SOX2, c-Myc, Klf4, and NANOG were measured by qRT-PCR in this experiment. Total RNA was isolated by using Trizol (Invitrogen) from the cells according to the manufacturer's instructions. Total RNA concentration was determined by the ultraviolet spectrophotometer and the equal quantity of RNA was reverse-transcribed into cDNA by using an M-MLV reverse transcriptase kit (GeneDireX). Real-time RT-PCR was performed using the QuantStudio™ 3 Real-time PCR system (ABI). The quantitative RT-PCR reactions contained 0.5 μM of each forward and reverse primer, lx SYBR green mix, and 2 μl cDNA. Amplification curves were generated with an initial denaturing step at 95° C. for 2 min, followed by 40 cycles of 95° C. for 5 s, 60° C. for 10 s and 72° C. for 15 s. The relative level of gene expression was normalized to glyceraldehyde 3-phosphate dehydrogenase and the fold change was calculated by the 2′. The results are shown as means±SD of three independent experiments each performed in triplicate. As shown in FIG. 1, the expressions of sternness-related genes OCT4, c-Myc, and NANOG in the transfected acinar cells (ACs/GnT-V) are significantly increased compared with control ACs/Mock. It is shown that GnT-V can significantly induce the expressions of the stem cell markers in acinar cells. In addition to determining the mRNA expression of ACs/GnT-V, this experiment further determined the actual expression of sternness-related surface proteins Sox2, Oct3/4, and Nanog on the cells by Flow cytometry. Aliquots of 5×10⁶ cells (ACs/Mock and ACs/GnT-V) were placed into a round bottom tube, washed with ice-cold PBS, and fixed with ice-cold 70% ethanol overnight. Fixed cells were incubated with 1 μM of primary antibody which was diluted in 3% BSA/PBS for 30 min at 4° C. and then washed three times with PBS. The experiment was analyzed by using FACSCalibur flow cytometer (BD Biosciences). The results are shown in FIG. 2. The levels of sternness-related Sox2, Oct3/4, and Nanog expression were increased on ACs/GnT-V surface, which is consistent with the results of mRNA determination. Besides, it has been reported that CD90/Thy-1 and CD49f/integrin α6 expression were positively correlated with sternness properties (Chen et al., International Journal of Oncology, 49, 1881-1889, 2016; Yu et al., Stem Cells, 30, 876-887, 2012). Therefore, ACs/Mock and ACs/GnT-V were stained in 2% FBS/PBS with primary antibodies, and were analyzed by using FACSCalibur flow cytometer (BD Biosciences) as well. The results are shown in FIG. 3. CD90/Thy-1 and CD49f/integrin α6 on the cell surface of ACs/GnT-V were significantly higher than those of ACs/Mock. It is shown that GnT-V can enhance CD90 and CD49f expression on ACs too. In addition, the protein expression levels of α-amylase, AQP3, and AQP5 were analyzed by western blot in this experiment for determining the salivary secretion ability of the transfected acinar cells (ACs/GnT-V). The whole cells lysate was prepared with RIPA lysis buffer (50 mM Tris-HCl, pH 7.4, 150 mM NaCl, 1% NP-40, 0.1% sodium dodecyl sulphate (SDS), and 0.5% sodium deoxychlolate) containing protease inhibitor cocktail (Roche Diagnostics GmbH), and then sonicated for 2 min and centrifuged at 13,300 rpm for 30 min at 4° C. The protein concentration was detected with a protein assay reagent (Bio-Rad). Then, an equal quantity of protein from cell lysates was resuspended with sample buffer, separated by SDS-polyacrylamide gel electrophoresis, and transferred to polyvinylidene fluoride membranes (Millipore). The membranes were blocked with blocking buffer for 30 min at room temperature and incubated with primary antibodies at 4° C. for overnight. After being washed and incubated with secondary antibodies, the protein expression was detected with ECL reagents and UVP system (Analytikjena). The result is shown in FIG. 4. Both ACs/GnT-V and ACs/Mock showed similar AQP3 expression, but the α-amylase and AQP5 expressions were more obvious in ACs/GnT-V than in ACs/Mock. Since AQP5 is the functional protein responsible for water permeability and α-amylase is the most important enzyme in saliva, GnT-V-overexpressing acinar cells enhance its sternness and increase the synthesis and secretion of salivary components. Furthermore, the proliferation ability of the transfected acinar cells (ACs/GnT-V) was determined by MTT assay in this experiment. MTT assay is used for detecting the activity of enzymes that reduce MTT to purple formazan in living cells. Briefly, the ACs/Mock and ACs/GnT-V were seeded into 96-well plates at a density of 1000 cells/per well to let the cells adhere overnight. After incubation for 24, 48, and 72 h, the MTT reagent (0.5 mg/ml) was added to each well and then incubated for an additional 4 h. At the end of the incubation period, the medium was moved and purple formazan was solubilized with dimethyl sulfoxide. The absorbance of the dye was measured at a wavelength of 570 nm with background subtraction at 630 nm by enzyme-linked immunoassay reader. FIGS. 5A-5C show the cell proliferation of different cell passages obtained by MTT assay between ACs/Mock and ACs/GnT-V (P1: passage 1; P2: passage 2; P3: passage 3). The results indicated that ACs/GnT-V and ACs/Mock exhibited different proliferation rates. The cell proliferation rates were similar in passage 3 compared to passage 1 in ACs/GnT-V but obviously decreased in ACs/Mocks with the increase in the number of passages. Moreover, ACs/Mock and ACs/GnT-V were serially passaged until the cells stopped dividing and the population doubling was detected by trypan blue assay. The result is shown in FIG. 6. ACs/Mock ceased dividing entirely at Day 20. Conversely, ACs/GnT-V maintained cell division ability to Day 80, indicating GnT-V expression positively correlated with cell proliferation in AC model. Finally, to ensure that overexpression of GnT-V promotes sternness rather than cancerization of the acinar cells, the mobility and invasiveness of ACs/GnT-V were observed. The results are shown in FIG. 7. GnT-V did not enhance the motility as well as invasiveness of ACs, indicating sternness but not cancerization was promoted in ACs/GnT-V. In conclusion, the sternness of the acinar cell can be promoted by using the method of the present application to overexpress GnT-V. The relevant mechanism has been understood from our other studies. The overexpression of GnT-V can activate the EGFR signaling pathway, increase the expression of ALDH1A3, and further increase the sternness of acinar cells. The details are described in the paper “Overexpression of N-acetylglucosaminyltransferase V promotes human parotid gland acinar cell immortalization via the epidermal receptor activation” by the inventors in J Cell Physiol. (237(3):1780-1789) in 2022. The contents of the paper are incorporated by reference in their entireties herein. Example 4—determinations of transdifferentiation of GnT-V-overexpressing acinar cells To investigate the possibility of transdifferentiation of salivary gland acinar cells into insulin-secreting cells, insulin in cellular of, and secreted by ACs/GnT-V were determined in this example. The expression in cellular of ACs/GnT-V was by western blot assay. The result is shown in FIG. 8. A high level of GnT-V significantly enhanced insulin protein expression in salivary gland ACs. Besides, insulin in the culture mediums was assayed by insulin ELISA kit (ab100578, Abcam biotech). The results are shown in FIG. 9. Compared to ACs/Mock, insulin concentration in the medium was significantly increased in ACs/GnT-V. Therefore, these findings suggest that the increment in GnT-V expression positively correlated with a large increase in insulin formation in salivary gland ACs. It indicates that GnT-V-overexpressing acinar cells can transdifferentiate into insulin-secreting cells. In addition, the conditioned mediums of ACs/Mock and ACs/GnT-V were collected in this experiment, and were determined whether they could activate the insulin receptor (IR) of HCT-116 to form a phosphorylated insulin receptor (pIR). The result is shown in FIG. 10. The conditioned medium of ACs/GnT-V enhanced the phosphorylation of IR in HCT116 after adding the conditioned medium for 10 minutes. Further, for the glucose uptake assay, the luminescence of HCT116 treated with conditioned mediums was evaluated with a general protocol of Glucose uptake-Glo (Promega, Wisconsin, United States) and ELISA reader in this experiment. The result is shown in FIG. 11. Glucose uptake of HCT116 was also significantly increased (p<0.05). The results confirmed that the insulin secreted by ACs/GnT-V was active and functional. In conclusion, the acinar cell can be promoted to transdifferentiate into insulin-secreting cells by using the method of the present application to overexpress GnT-V. The relevant mechanism has been understood from our other studies. The overexpression of GnT-V can activate the EGFR signaling pathway, increase the expression of CPH, promote cellular proinsulin to convert to insulin and C-peptide, and further promote the secretion of insulin.

What is claimed is:

1. A method for promoting the sternness and/or transdifferentiation of acinar cells, comprising the following steps:
providing an acinar cell,
transfecting a plasmid into the acinar cell, and
culturing the transfected acinar cell,
wherein the plasmid contains a genetic material for overexpression of N-acetylglucosaminyltransferase V (GnT-V).

2. The method of claim 1, wherein the plasmid is transfected into the acinar cell by calcium phosphate, liposome, viral vector or electroporation.

3. The method of claim 2, wherein the viral vector is lentiviral vector.

4. The method of claim 1, wherein the genetic material contains MGAT5 cDNA, the sequence set forth as SEQ ID NO: 1.

5. The method of claim 4, wherein the MGAT5 cDNA is derived by amplifying with primers of SEQ ID NO: 2 and SEQ ID NO: 3.

6. The method of claim 1, wherein the transfected acinar cell can transdifferentiate into an insulin-secreting cell.

7. The method of claim 1, wherein the acinar cell is a salivary gland acinar cell.

8. The method of claim 7, wherein the salivary gland acinar cell is a parotid gland acinar cell.

9. An insulin-secreting cell obtained by using the method of claim 1.

10. A method of insulin production, comprising all the steps of claim 1, and further comprising a step of harvesting insulin from the cell culture medium.
